(19) United States
(12) Patent Application Publication
(10) Pub. No.: US 2024/0350568 A1
(43) Pub. Date: Oct. 24, 2024

(54) NATURAL WATER-SOLUBLE PLANT COMPOUND WITH ANTIDEPRESSANT AND SLEEP-PROMOTING EFFECTS, AND PREPARATION METHOD AND USE THEREOF

(71) Applicant: LVLH Tech Holdings LLC, Henderson, NV (US)

(72) Inventors: Jiangxin WANG, Shenzhen (CN); Shiyu XU, Shenzhen (CN)

(21) Appl. No.: 18/221,977

(22) Filed: Jul. 14, 2023

(30) Foreign Application Priority Data
Apr. 21, 2023 (CN) 2023104639598

(51) Int. Cl. A61K 36/05; A61K 35/68; A61K 36/48; A61K 36/725; A61K 36/815; A61K 36/82; A61K 36/896; A61K 36/899; A61P 25/20; A61P 25/24

(52) U.S. Cl. CPC A61K 36/05; A61K 35/68; A61K 36/48; A61K 36/725; A61K 36/815; A61K 36/82; A61K 36/896; A61K 36/899; A61P 25/20; A61P 25/24

(57) ABSTRACT

The present disclosure relates to a natural water-soluble plant compound with antidepressant and sleep-promoting effects, and a preparation method and use thereof. In the present disclosure, the preparation method includes: immersing Fructus lycii, green tea, spina date seed, lily, sugarcane, peanut stem, Euglena, and Chlorella in water, and conducting ultrasonication and first solid-liquid separation to obtain a plant extract; mixing the plant extract with an organic extractant to extract an extracted aqueous phase; removing a solvent of the extracted aqueous phase to obtain a crude extract; dispersing the crude extract into water, and conducting second solid-liquid separation to obtain a purified water extract; and drying the purified water extract to obtain the natural water-soluble plant compound. The natural water-soluble plant compound shows a wide range of neuroactivity, and has an important broad-spectrum improvement effect on alleviating various discomforts caused by the fast-paced and high-stress life of modern people.

### CROSS REFERENCE TO RELATED APPLICATION

This patent application claims the benefit and priority of Chinese Patent Application No. 2023104639598, filed with the China National Intellectual Property Administration on Apr. 21, 2023, the disclosure of which is incorporated by reference herein in its entirety as part of the present application.

### TECHNICAL FIELD

The present disclosure belongs to the technical field of plant extraction, and in particular relates to a natural water-soluble plant compound with antidepressant and sleep-promoting effects, and a preparation method and use thereof.

### BACKGROUND

Sleep plays an integral role in health. To restore optimal brain and body health, everyone must get enough sleep. The regulation of sleep is divided into two aspects: circadian rhythm and sleep homeostasis. Sleep pressure gradually increases with longer waking hours; as sleep progresses, the sleep pressure is gradually cleared. The circadian rhythm controls the time of sleep and wake during the day through an internal biological clock, and is mainly mediated by melatonin. The sleep homeostasis enables the body to obtain a certain amount of sleep mainly by regulating the sleep pressure. However, in the high-paced and high-competition modern society, sleep seems to have become a luxury. Lack of sleep has become a global epidemic. According to statistics, 30% to 40% of global populations suffer from sleep problems. About 38% of Chinese adults have insomnia. Currently, sleep hormones on the market include 4 categories: single melatonin, sedatives, sleeping pills, and comprehensive drugs.

On the other hand, according to the World Health Organization (WHO), more than 350 million people worldwide suffer from depression. Depression has become the fourth leading disease in the world and is growing rapidly. The WHO predicts that depression may rank first in the global disease burden by 2030. Research shows that major depressive disorder and anxiety disorder have increased by 28% and 26% globally in 2020, respectively. So far, commonly-used anti-anxiety drugs are mainly benzodiazepines, which are characterized by strong action, quick onset, safety, and reliability. Of course, these drugs also have side effects, commonly including drowsiness, dizziness, fatigue, lethargy, and constipation. High dosages of these drugs may cause tremor, blurred vision, irritability, insomnia, and skin rashes.

Insomnia and depression have many causes and can lead to a huge impact on an individual's health. However, the current amelioration and treatment methods are limited, and there are few types of drugs that can simultaneously improve the insomnia and treat the depression.

### SUMMARY

An objective of the present disclosure is to provide a sleep repair factor-containing nutrient solution, namely a natural water-soluble plant compound with antidepressant and sleep-promoting effects, and a preparation method and use thereof. In the present disclosure, a ratio of each principal component is optimized through years of research results on Dynamic Regulation Network of Multiple Targets (DRNMT), which is combined with biotechnology (BT) and artificial intelligence (AI). The sleep repair factor-containing nutrient solution has a particularly unexpected effect within the proportioning range, and shows antidepressant and sleep-promoting effects, regulates neuroactivity, and shows a wide range of applications. The compound has an important broad-spectrum improvement effect on relieving insomnia and depression brought about by the fast-paced and high-stress life of modern people. Meanwhile, the sleep repair factor-containing nutrient solution (natural water-soluble plant compound) has unique natural plant fragrance and excellent water solubility.

To achieve the above objective, the present disclosure provides the following technical solutions:

The present disclosure provides a preparation method of a natural water-soluble plant compound, including the following steps:

immersing a plant raw material in water, and conducting ultrasonication and first solid-liquid separation to obtain a plant extract; where the plant raw material includes Fructus lycii, green tea, spina date seed, lily, sugarcane, peanut stem, Euglena, and Chlorella;
mixing the plant extract with an organic extractant to extract an extracted aqueous phase;
removing a solvent of the extracted aqueous phase to obtain a crude extract;
dispersing the crude extract into water, and conducting second solid-liquid separation to obtain a purified water extract; and
drying the purified water extract to obtain the natural water-soluble plant compound.

Preferably, the plant raw material includes the following components in parts by mass:

20 parts to 50 parts of the Fructus lycii, 15 parts to 40 parts of the green tea, 15 parts to 30 parts of the spina date seed, 10 parts to 20 parts of the lily, 5 parts to 20 parts of the sugarcane, 3 parts to 10 parts of the peanut stem, 1 part to 10 parts of the Euglena, and 1 part to 10 parts of the Chlorella.

Preferably, the organic extractant is ethyl acetate; and the plant extract and the organic extractant are at a volume ratio of 1:(3-4).

Preferably, the solvent is removed by rotary evaporation at 60° C. to 65° C. and 9 rpm to 10 rpm.

Preferably, the ultrasonication is conducted in an ice-water bath at an ultrasonic power of 700 W to 1,000 W for 8 min to 10 min.

Preferably, the drying refers to freeze-drying at −80° C. to −70° C. for 30 min to 40 min.

Preferably, the first solid-liquid separation includes first centrifugal separation and membrane filtration that are sequentially conducted;
the first centrifugal separation is conducted at a rotational speed of 4,000 g to 5,000 g for 5 min to 8 min; and the membrane filtration is conducted using a filter membrane with a pore size of 0.22 μm; and
the second solid-liquid separation is conducted by second centrifugal separation at a rotational speed of 8,000 g to 10,000 g for 10 min.

The present disclosure further provides a natural water-soluble plant compound prepared by the preparation method, including y-aminobutyric acid, phenylacetic acid, glutamic acid, glycine, and taurine.

Preferably, the natural water-soluble plant compound includes (1-200) μg/100 mg of the y-aminobutyric acid, (0.1-50) μg/100 mg of the phenylacetic acid, (0.1-20) μg/100 mg of the glutamic acid, (0.01-8) μg/100 mg of the glycine, and (0.01-0.2) μg/100 mg of the taurine.

The present disclosure further provides use of the natural water-soluble plant compound in preparation of a drug or a health care product for treating insomnia and depression.

The present disclosure provides a preparation method of a sleep repair factor-containing nutrient solution (natural water-soluble plant compound), including the following steps: immersing a plant raw material in water, and conducting ultrasonication and first solid-liquid separation to obtain a plant extract; where the plant raw material includes Fructus lycii, green tea, spina date seed, lily, sugarcane, peanut stem, Euglena, and Chlorella; mixing the plant extract with an organic extractant to extract an extracted aqueous phase; removing a solvent of the extracted aqueous phase to obtain a crude extract; dispersing the crude extract into water, and conducting second solid-liquid separation to obtain a purified water extract; and drying the purified water extract to obtain the natural water-soluble plant compound. In the present disclosure, the preparation method uses Fructus lycii, green tea, spina date seed, lily, sugarcane, peanut stem, Euglena, and Chlorella as raw materials, and conducts ultrasonication, solid-liquid separation, extraction, solvent removal, secondary water dissolution, and drying in sequence. In this way, natural water-soluble compounds can be efficiently extracted from the Fructus lycii, green tea, spina date seed, lily, sugarcane, peanut stem, Euglena, and Chlorella. The results of animal experiments in the examples prove that the natural water-soluble plant compound has both antidepressant and sleep-promoting activities, and shows unique neuroactivity and dosage relationship. The natural water-soluble plant compound shows a wide range of neuroactivity, and has an important broad-spectrum improvement effect on alleviating various discomforts caused by the fast-paced and high-stress life of modern people. Meanwhile, the natural water-soluble plant compound has unique natural plant fragrance and excellent water solubility, and can be taken alone or used as a nutritional supplement.

### BRIEF DESCRIPTION OF THE DRAWINGS

FIG. 1 shows an effect of the natural water-soluble plant compound prepared by Example 1 of the present disclosure on sleep time;

FIG. 2 shows an effect of products prepared by Comparative Examples 1 to 3 of the present disclosure on sleep time;

FIG. 3 shows an effect of the natural water-soluble plant compound prepared by Example 1 of the present disclosure on barbital sodium-induced sleep latency in mice;

FIG. 4 shows an effect of the products prepared by Comparative Examples 1 to 3 of the present disclosure on barbital sodium-induced sleep latency in mice;

FIG. 5 shows an effect of the natural water-soluble plant compound prepared by Example 1 of the present disclosure on non-swimming time; and

FIG. 6 shows an effect of the products prepared by Comparative Examples 1 to 3 of the present disclosure on non-swimming time.

### DETAILED DESCRIPTION OF THE EMBODIMENTS

The present disclosure provides a preparation method of a natural water-soluble plant compound, including the following steps:
immersing a plant raw material in water, and conducting ultrasonication and first solid-liquid separation to obtain a plant extract; where the plant raw material includes Fructus lycii, green tea, spina date seed, lily, sugarcane, peanut stem, Euglena, and Chlorella;
mixing the plant extract with an organic extractant to extract an extracted aqueous phase;
removing a solvent of the extracted aqueous phase to obtain a crude extract;
dispersing the crude extract into water, and conducting second solid-liquid separation to obtain a purified water extract; and
drying the purified water extract to obtain the natural water-soluble plant compound.

In the present disclosure, unless otherwise specified, all raw materials/components for preparation are commercially available products well known to those skilled in the art.

In the present disclosure, a plant raw material is immersed in water, and ultrasonication and first solid-liquid separation are conducted to obtain a plant extract; where the plant raw material includes Fructus lycii, green tea, spina date seed, lily, sugarcane, peanut stem, Euglena, and Chlorella.

In the present disclosure, the plant raw material includes preferably the following components in parts by mass: 20 parts to 50 parts of the Fructus lycii, 15 parts to 40 parts of the green tea, 15 parts to 30 parts of the spina date seed, 10 parts to 20 parts of the lily, 5 parts to 20 parts of the sugarcane, 3 parts to 10 parts of the peanut stem, 1 part to 10 parts of the Euglena, and 1 part to 10 parts of the Chlorella, more preferably 22 parts to 47 parts of the Fructus lycii, 16 parts to 35 parts of the green tea, 18 parts to 26 parts of the spina date seed, 12 parts to 19 parts of the lily, 6 parts to 17 parts of the sugarcane, 4 parts to 9 parts of the peanut stem, 2 parts to 8 parts of the Euglena, and 2 parts to 8 parts of the Chlorella.

In the present disclosure, the Fructus lycii is preferably fresh Fructus lycii, and the green tea is preferably green tea leaves.

In the present disclosure, the plant raw material is preferably washed before the immersing. The washing preferably includes conducting water washing on each component of the plant raw material separately. The water washing is preferably conducted by deionized water preferably 3 times at preferably 4° C. During each water washing, solid-liquid separation is preferably conducted by centrifugal separation at a rotational speed of preferably 8,000 g to 10,000 g for preferably 3 min.

In the present disclosure, the water is preferably deionized water.

In the present disclosure, there is no special requirement on an amount of the water, as long as the plant raw material of compound is completely submerged in water.

Preferably, the ultrasonication is preferably conducted in an ice-water bath at an ultrasonic power of preferably 700 W to 1,000 W, more preferably 700 W for preferably 8 min to 10 min, more preferably 8 min. The ultrasonication preferably adopts an intermittent working mode, and the intermittent working mode is preferably conducted in a cycle including 3 s of ultrasound and 4 s of off-ultrasound.

In the present disclosure, a crushed feed liquid is obtained by the ultrasonication, and the first solid-liquid separation is conducted on the crushed feed liquid.

In the present disclosure, the first solid-liquid separation includes preferably first centrifugal separation and membrane filtration that are sequentially conducted. The first centrifugal separation is conducted at preferably 4° C. and a rotational speed of preferably 4,000 g to 5,000 g, more preferably 4,000 g for preferably 5 min to 8 min, more preferably 5 min. The membrane filtration is preferably conducted using a filter membrane with a pore size of 0.22 μm.

In the present disclosure, the plant extract is mixed with an organic extractant to extract an extracted aqueous phase.

In the present disclosure, the organic extractant is preferably ethyl acetate.

In the present disclosure, the plant extract and the organic extractant are at a volume ratio of preferably 1:(3-4), more preferably 1:3.

In the present disclosure, the extraction is preferably conducted in a separatory funnel, and includes preferably mixing the plant extract and the organic extractant and then allowing to stand for 20 min.

In the present disclosure, a solvent of the extracted aqueous phase is removed to obtain a crude extract.

In the present disclosure, the solvent is removed by rotary evaporation at preferably 60° C. to 65° C., more preferably 65° C. and a rotational speed of preferably 9 rpm to 10 rpm.

In the present disclosure, the rotary evaporation is preferably conducted in a rotary evaporator.

In the present disclosure, the crude extract is dispersed into water, and second solid-liquid separation is conducted to obtain a purified water extract.

In the present disclosure, the second solid-liquid separation is preferably conducted by second centrifugal separation at a rotational speed of preferably 8,000 g to 10,000 g, more preferably 8,000 g for preferably 10 min.

In the present disclosure, the purified water extract is dried to obtain the natural water-soluble plant compound.

In the present disclosure, the drying is preferably freeze-drying at preferably −80° C. to −70° C., more preferably −80° C. for preferably 30 min to 40 min, more preferably 30 min.

The present disclosure further provides a natural water-soluble plant compound prepared by the preparation method, including y-aminobutyric acid, phenylacetic acid, glutamic acid, glycine, and taurine.

In the present disclosure, the natural water-soluble plant compound is a dry powder.

In the present disclosure, the natural water-soluble plant compound includes (1-200) μg/100 mg of the y-aminobutyric acid, (0.1-50) μg/100 mg of the phenylacetic acid, (0.1-20) μg/100 mg of the glutamic acid, (0.01-8) μg/100 mg of the glycine, and (0.01-0.2) μg/100 mg of the taurine.

In the present disclosure, in parts by mass, the plant raw material including 20 parts to 50 parts of the fresh Fructus lycii, 15 parts to 40 parts of the green tea leaves, 15 parts to 30 parts of the spina date seed, 10 parts to 20 parts of the lily, 5 parts to 20 parts of the sugarcane, 3 parts to 10 parts of the peanut stem, 1 part to 10 parts of the Euglena, and 1 part to 10 parts of the Chlorella are subjected to ultrasonication, separation and collection, extraction with ethyl acetate, rotary evaporation, water dissolution, and freeze-drying in sequence. In this way, specific natural compounds in these plants can be extracted in a targeted and efficient manner, and finally a dry powder is prepared. The natural water-soluble plant compound includes y-aminobutyric acid, phenylacetic acid, glutamic acid, glycine, and taurine.

The present disclosure further provides use of the natural water-soluble plant compound in preparation of a drug or a health care product for treating insomnia and depression.

In the present disclosure, animal experiments prove that the natural water-soluble plant compound has both antidepressant and sleep-promoting activities, and shows unique neuroactivity and dosage relationship. This natural water-soluble plant compound shows a wide range of neuroactivity, and has an important broad-spectrum improvement effect on alleviating various discomforts caused by the fast-paced and high-stress life of modern people. Meanwhile, this powder has unique natural plant fragrance and excellent water solubility, and can be taken alone or used as a nutritional supplement.

In order to further illustrate the present disclosure, the technical solutions provided by the present disclosure will be described in detail below in conjunction with accompanying drawings and examples, but they should not be construed as limiting the protection scope of the present disclosure.

### Example 1

In this example, a plant raw material included: 25 parts of fresh Fructus lycii, 20 parts of green tea leaves, 15 parts of spina date seed, 20 parts of lily, 20 parts of sugarcane, 8 parts of peanut stem, 3 parts of Euglena, and 3 parts of Chlorella,
the plant raw material was washed by centrifugation 3 times with deionized water (replenished to 50 mL) at 10,000 g and 4° C. for 3 min; and
an obtained centrifuged and clear plant raw material was collected in one tube, added with 20 mL of deionized water (just to submerge the plant raw material, precautions:when a resulting mixed solution was up to 2 L, materials larger than 2 L needed to be crushed in batches); a resulting mixture was subjected to ultrasonication in an ice-water bath at an ultrasonic power of 700 W for 8 min, with a cycle including 3 s of ultrasound and 4 s of off-ultrasound.

An obtained feed solution after the ultrasonication was subjected to centrifugal separation at 4° C. and 5,000 g for 5 min, a precipitate (the precipitate contained broken cells and insoluble matters) was discarded, and an obtained supernatant was collected; the supernatant was an opaque yellow-green liquid, or a brown transparent liquid; and
the supernatant was subjected to membrane filtration with a 0.22 μm filter membrane (which could be operated outdoors) to obtain an extract.

According to a volume ratio of extract: ethyl acetate=1:3, the extract and the ethyl acetate were mixed in a clean Erlenmeyer flask to obtain a mixed solution. An inner wall of a separatory funnel was washed with deionized water before use, and then airtightness of the separatory funnel was tested with about 50 mL of absolute ethanol. If the airtightness was desirable, the separatory funnel was washed with deionized water. The mixed solution was poured into the separatory funnel and allowed to stand for 20 min until layers were separated. An upper white layer was an organic layer, and a lower yellow layer was an aqueous phase, and a solution of the aqueous phase was collected in a clean Erlenmeyer flask.

The solution of the aqueous phase was subjected to rotary evaporation. Before the rotary evaporation, an entire rotary evaporator was washed once with a methanol solution, that is, the rotary evaporation was conducted once. The rotary evaporation with methanol was conducted in a water bath at 29° C. and a rotational speed of a rotary bottle of 7 rpm to 8 rpm. An obtained extracted solution of the aqueous phase was poured into the rotary bottle (with a volume not exceeding 50% that of the rotary bottle), and subjected to rotary evaporation at 65° C. and 9 rpm to 10 rpm. The rotary evaporation was conducted until a solution in the rotary bottle was solid. The rotary evaporation was terminated.

The solid on the wall was eluted with 1 mL of deionized water each time, and the solid was a crude water-soluble substance at this time, and an obtained crude product solution was brown.

The crude product solution was centrifuged at 10,000 g for 10 min, and an obtained supernatant was transferred into a pre-weighed ep tube to obtain a purified solution.

The ep tube was uncapped, a mouth of the tube was sealed with a parafilm, several small openings were poked with a needle, and the purified solution was frozen at −80° C. for 30 min; a resulting frozen solution was quickly transferred to a freeze-dryer for overnight drying, to obtain a powder of a natural water-soluble plant compound.

The active components in the powder of the natural water-soluble plant compound prepared in Example 1 were quantitatively determined by HPLC.

100 mg of the powder of the natural water-soluble plant compound was dissolved in 5 mL of distilled water, and the determination of related substances was conducted by HPLC. The powder of the natural water-soluble plant compound included: y-aminobutyric acid (GABA) (40 μg/100 mg), phenylacetic acid (10 μg/100 mg), glutamic acid (4 μg/100 mg), glycine (1 μg/100 mg), taurine (0.1 μg/100 mg), and artificial products pentobarbital (0) and duloxetine (0).

### Comparative Example 1

A preparation method was basically the same as that in Example 1, except that the plant raw material included: 25 parts of the fresh Fructus lycii, 20 parts of the green tea leaves, and 15 parts of the spina date seed, and an obtained product was recorded as a product A.

### Comparative Example 2

A preparation method was basically the same as that in Example 1, except that the plant raw material included: 20 parts of the lily, 20 parts of the sugarcane, and 8 parts of the peanut stem, and an obtained product was recorded as a product B.

### Comparative Example 3

A preparation method was basically the same as that in Example 1, except that the plant raw material included: 3 parts of Euglena and 3 parts of Chlorella, and an obtained product was recorded as a product C.

### Example 2

A preparation method was basically the same as that in Example 1, except that the plant raw material included: 45 parts of the fresh Fructus lycii, 35 parts of the green tea leaves, 30 parts of the spina date seed, 10 parts of the lily, 5 parts of the sugarcane, 3 parts of the peanut stem, 10 parts of the Euglena, and 10 parts of the Chlorella.

### Example 3

A preparation method was basically the same as that in Example 1, except that the plant raw material included: 20 parts of the fresh Fructus lycii, 40 parts of the green tea leaves, 20 parts of the spina date seed, 15 parts of the lily, 20 parts of the sugarcane, 10 parts of the peanut stem, 8 parts of the Euglena, and 8 parts of the Chlorella.

### Test Example 1

The experiment of prolonging sleep time of pentobarbital sodium of the powder of the natural water-soluble plant compound prepared by Example 1

Experimental animals: Kunming SPF-grade male mice weighing 20 g. In Test Examples 1 to 3, the mice were randomly divided into 10 groups in each experiment, with 15 mice in each group, and the test time was 30 d.

Before the experiment, a dosage of pentobarbital sodium (40 mg/kg) that made the animals fall asleep 100% but did not make the sleep time too long was determined, and the formal experiment was conducted with same dosage. A sample of the powder of the natural water-soluble plant compound prepared in Example 1 was weighed, and mixed with purified water to prepare a 50 mL sample solution for gavage. The tested sample solution was orally gavaged by 0.1 mL/10 g bw every day. The final contents were 5 mg/20 g, 15 mg/20 g, and 30 mg/20 g of the compound powder, which were a low dosage, a medium dosage, and a high dosage, respectively.

The samples prepared in Comparative Examples 1 to 3 were prepared with purified water to obtain 50 mL of a sample solution for gavage. The tested sample solution was orally gavaged by 0.1 mL/10 g bw every day. The final content was 15 mg/20 g of the sample, which was a medium dosage.

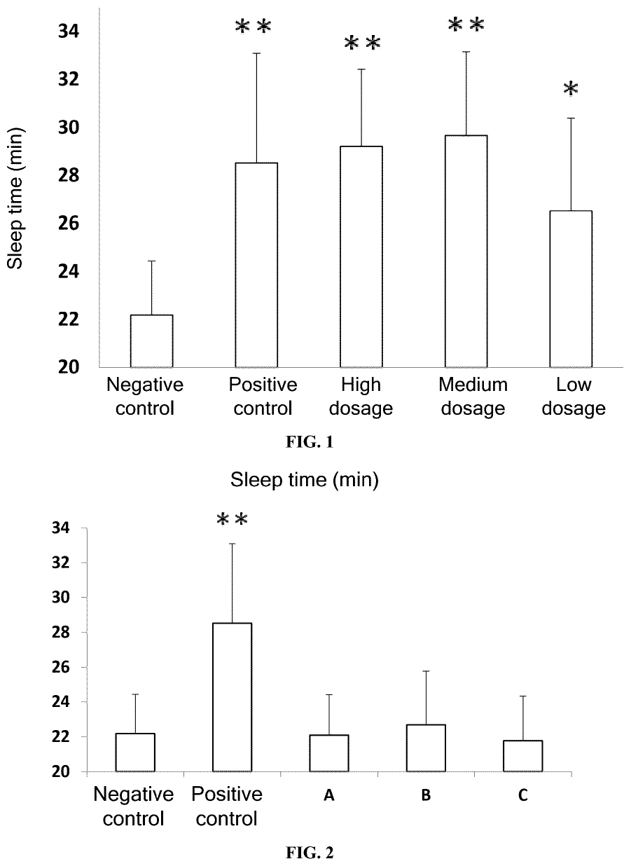

3 mg of melatonin was used as a positive control. 15 min after the animals were given the test samples of the control group and the test group for the last time, the animals in each group were intraperitoneally injected with 40 mg/kg bw of pentobarbital sodium at an injection volume of 0.1 mL/10 g bw. Taking the disappearance of righting reflex as an index, whether the test sample could prolong the sleep time induced by pentobarbital sodium was observed. The obtained data were subjected to variance analysis with SPSS. The test results were shown in FIG. 1 and FIG. 2.

FIG. 1 showed an effect of the compound powder prepared in Example 1 on sleep time (*, p<0.05; **, p<0.01). FIG. 1 showed the effect of each group on the sleep time of mice induced by pentobarbital sodium. The experimental results in FIG. 1 showed that the sleep time of the animals in each test group was prolonged, and there was a significant difference in the sleep time compared with the positive control group, and the effect of the medium dosage was more significant. This indicated that the compound powder prepared in Example 1 of the present disclosure prolonged the sleep time induced by pentobarbital sodium, and the test result was positive.

Meanwhile, 3 groups of mice were also tested, which were only given the products prepared in Comparative Examples 1 to 3 at medium dosages, and the test results were shown in FIG. 2. FIG. 2 showed the effect of the medium dosage (10 mg/20 g) of Comparative Example 1 (A), Comparative Example 2 (B), and Comparative Example 3 (C) on the sleep time (*, p<0.05; **, p<0.01). As shown in FIG. 2, the products prepared in Comparative Examples 1 to 3 did not have any significant difference in increasing the sleep time.

### Test Example 2

### Experiment of Sleep Latency of Barbital Sodium

Before the formal experiment, a pre-test was conducted to determine a dosage of barbital sodium (200 mg/kg) that made the animals fall asleep 100% but did not make the sleep time too long, and the formal experiment was conducted with same dosage. A sample of the powder of the natural water-soluble plant compound prepared in Example 1 was weighed, and mixed with purified water to prepare a 50 mL sample solution for gavage. The tested sample solution was orally gavaged by 0.1 mL/10 g bw every day. The final contents were 5 mg/20 g, 15 mg/20 g, and 30 mg/20 g of the compound powder, which were a low dosage, a medium dosage, and a high dosage, respectively.

The samples prepared in Comparative Examples 1 to 3 were prepared with purified water to obtain 50 mL of a sample solution for gavage. The tested sample solution was orally gavaged by 0.1 mL/10 g bw every day. The final content was 15 mg/20 g of the sample, which was a medium dosage.

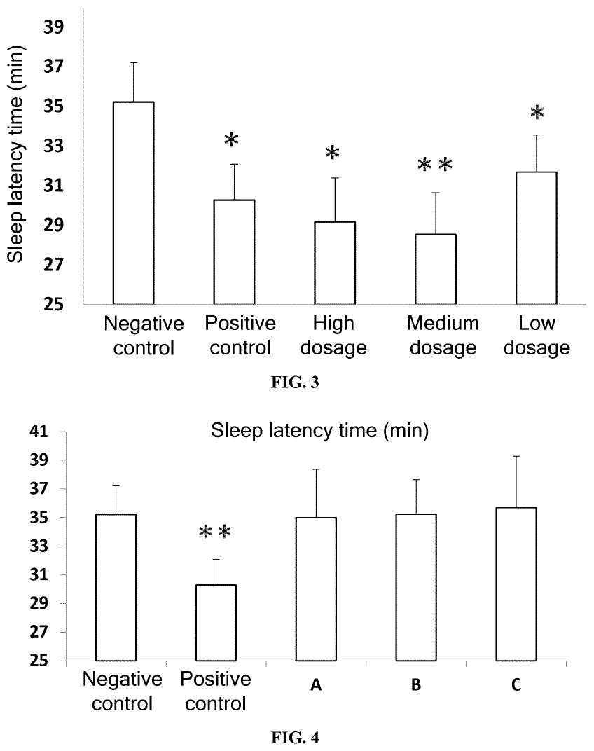

3 mg of melatonin was used as a positive control. 15 min after the animals were given the test samples of the control group and the test group for the last time, the animals in each group were intraperitoneally injected with barbital sodium at an injection volume of 0.1 mL/10 g bw. Taking the disappearance of righting reflex as an index, the effect of the test sample on the sleep latency of barbital sodium was observed. The obtained data were subjected to variance analysis with SPSS. Experimental results were shown in FIG. 3. FIG. 3 showed the effects of the compound powders of each group in Example 1 on the sleep latency of mice induced by barbital sodium (*, p<0.05; **, p<0.01). As shown in FIG. 3, the experimental results of the effect of the compound powder prepared in Example 1 on barbital sodium-induced sleep latency in mice showed that: in each test group, the sleep latency was shortened, and the sleep latency was significantly different from that of the negative control. This indicated that the experimental result of the barbital sodium sleep latency of the compound powder was positive.

Meanwhile, 3 groups of mice were also tested, which were only given the products prepared in Comparative Examples 1 to 3 at medium dosages, and the test results were shown in FIG. 4. FIG. 4 showed the effects of the compound powders of each group in Comparative Examples 1 to 3 on the sleep latency of mice induced by barbital sodium, (*, p<0.05; **, p<0.01). As shown in FIG. 4, the products prepared in Comparative Examples 1 to 3 had no significant difference in reducing the sleep latency compared with that of the negative control group.

### Test Example 3

Forced swimming test (FST): the FST is a reliable experimental method for screening and observing antidepressant drugs in experiments of behavioral pharmacology. The FST is a model that utilizes an animal's inability to escape a hostile environment that leads to animal behavior of despair. In the FST, an animal is placed in a container of water and forced to swim. The animal's initial response is to swim and try to escape, and then cease to struggle and swim after realizing that escape is impossible. At this time, the animal only keeps its head out of the water, with limbs floating, and maintains a motionless state, which is called “behavioral desperation”.

The FST included the following steps: the sample of the powder of the natural water-soluble plant compound prepared in Example 1 was weighed separately, and mixed with purified water to obtain 50 mL of three sample solutions, which were 3 dosages of 5 mg/20 g, 15 mg/20 g, and 30 mg/20 g, and denoted as a low dosage, a medium dosage, and a high dosage, respectively;
the samples of the powders of the natural water-soluble plant compounds prepared in Comparative Examples 1 to 3 were weighed, and mixed with purified water to prepare 50 mL of three sample solutions, which were 3 dosages of 15 mg/20 g, and denoted as a medium dosage; and
a blank control group (solvent) and a positive control group of duloxetine (10 mg/kg) were set up.

The test drug was added to water according to the set dosage, and the tested mice were orally administered at 10 mL/kg. After 1 h, the mice were put into an open cylindrical water tank with a diameter of 10 cm and a height of 25 cm, and water inside the tank was 20 cm high and at 25° C.±1° C. A video was recorded for 6 min, and an accumulated non-swimming time of the mice was recorded within 4 min. The non-swimming time of the mice could be understood as a reflection of the depression caused by the animal model, and the experimental results were sorted out by SMART3.0 animal experiment analysis software.

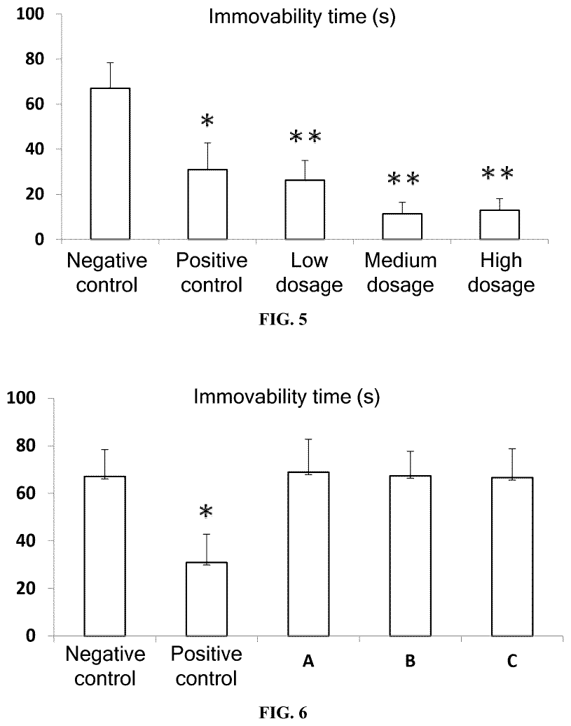

The experimental results showed that the three dosages of compound powder prepared in Example 1 could significantly shorten the immovability time of the mice in the tail suspension test and the FST. That is, the powder could weaken the state of “behavioral desperation” in mice and achieve an antidepressant effect, and the specific antidepressant effect was shown in FIG. 5. FIG. 5 showed the effect of the compound powder prepared in Example 1 on the non-swimming time (*, p<0.05; **, p<0.01).

The experimental results of Comparative Examples 1 to 3 were shown in FIG. 6. FIG. 6 showed the effect of the compound powders with absence prepared in Comparative Examples 1 to 3 on the non-swimming time (*, p<0.05; **, p<0.01). As shown in FIG. 6, none of the 3 compound powders with absence could significantly shorten the immovability time of the mice in the tail suspension test and FST. That is, the powder did not weaken the state of “behavioral desperation” in mice, and did not achieve the antidepressant effect.

The effect of the products prepared in Examples 2 to 3 was basically the same as that of the product prepared in Example 1.

Although the present disclosure is described in detail in conjunction with the foregoing examples, they are only a part of, not all of, the examples of the present disclosure. Other examples can be obtained based on these examples without creative efforts, and all of these examples shall fall within the protection scope of the present disclosure.

1. A preparation method of a natural water-soluble plant compound, comprising the following steps:
immersing a plant raw material in water, and conducting ultrasonication and first solid-liquid separation to obtain a plant extract; wherein the plant raw material comprises Fructus lycii, green tea, spina date seed, lily, sugarcane, peanut stem, Euglena, and Chlorella;
mixing the plant extract with an organic extractant to extract an extracted aqueous phase;
removing a solvent of the extracted aqueous phase to obtain a crude extract;
dispersing the crude extract into water, and conducting second solid-liquid separation to obtain a purified water extract; and
drying the purified water extract to obtain the natural water-soluble plant compound.

2. The preparation method according to claim 1, wherein the plant raw material comprises the following components in parts by mass:
20 parts to 50 parts of the Fructus lycii, 15 parts to 40 parts of the green tea, 15 parts to 30 parts of the spina date seed, 10 parts to 20 parts of the lily, 5 parts to 20 parts of the sugarcane, 3 parts to 10 parts of the peanut stem, 1 part to 10 parts of the Euglena, and 1 part to 10 parts of the Chlorella.

3. The preparation method according to claim 1, wherein the organic extractant is ethyl acetate; and the plant extract and the organic extractant are at a volume ratio of 1:(3-4).

4. The preparation method according to claim 1, wherein the solvent is removed by rotary evaporation at 60° C. to 65° C. and 9 rpm to 10 rpm.

5. The preparation method according to claim 1, wherein the ultrasonication is conducted in an ice-water bath at an ultrasonic power of 700 W to 1,000 W for 8 min to 10 min.

6. The preparation method according to claim 1, wherein the drying refers to freeze-drying at −80° C. to −70° C. for 30 min to 40 min.

7. The preparation method according to claim 1, wherein the first solid-liquid separation comprises first centrifugal separation and membrane filtration that are sequentially conducted;
the first centrifugal separation is conducted at a rotational speed of 4,000 g to 5,000 g for 5 min to 8 min; and the membrane filtration is conducted using a filter membrane with a pore size of 0.22 μm; and
the second solid-liquid separation is conducted by second centrifugal separation at a rotational speed of 8,000 g to 10,000 g for 10 min.

8. A natural water-soluble plant compound prepared by the preparation method according to claim 1, comprising y-aminobutyric acid, phenylacetic acid, glutamic acid, glycine, and taurine.

9. The natural water-soluble plant compound according to claim 8, wherein the plant raw material comprises the following components in parts by mass:
20 parts to 50 parts of the Fructus lycii, 15 parts to 40 parts of the green tea, 15 parts to 30 parts of the spina date seed, 10 parts to 20 parts of the lily, 5 parts to 20 parts of the sugarcane, 3 parts to 10 parts of the peanut stem, 1 part to 10 parts of the Euglena, and 1 part to 10 parts of the Chlorella.

10. The natural water-soluble plant compound according to claim 8, wherein the organic extractant is ethyl acetate; and the plant extract and the organic extractant are at a volume ratio of 1:(3-4).

11. The natural water-soluble plant compound according to claim 8, wherein the solvent is removed by rotary evaporation at 60° C. to 65° C. and 9 rpm to 10 rpm.

12. The natural water-soluble plant compound according to claim 8, wherein the ultrasonication is conducted in an ice-water bath at an ultrasonic power of 700 W to 1,000 W for 8 min to 10 min.

13. The natural water-soluble plant compound according to claim 8, wherein the drying refers to freeze-drying at −80° C. to −70° C. for 30 min to 40 min.

14. The natural water-soluble plant compound according to claim 8, wherein the first solid-liquid separation comprises first centrifugal separation and membrane filtration that are sequentially conducted;
the first centrifugal separation is conducted at a rotational speed of 4,000 g to 5,000 g for 5 min to 8 min; and the membrane filtration is conducted using a filter membrane with a pore size of 0.22 μm; and
the second solid-liquid separation is conducted by second centrifugal separation at a rotational speed of 8,000 g to 10,000 g for 10 min.

15. The natural water-soluble plant compound according to claim 8, comprising (1-200) μg/100 mg of the y-aminobutyric acid, (0.1-50) μg/100 mg of the phenylacetic acid, (0.1-20) μg/100 mg of the glutamic acid, (0.01-8) μg/100 mg of the glycine, and (0.01-0.2) μg/100 mg of the taurine.

16. The natural water-soluble plant compound according to claim 9, comprising (1-200) μg/100 mg of the y-aminobutyric acid, (0.1-50) μg/100 mg of the phenylacetic acid, (0.1-20) μg/100 mg of the glutamic acid, (0.01-8) μg/100 mg of the glycine, and (0.01-0.2) μg/100 mg of the taurine.

17. The natural water-soluble plant compound according to claim 10, comprising (1-200) μg/100 mg of the y-aminobutyric acid, (0.1-50) μg/100 mg of the phenylacetic acid, (0.1-20) μg/100 mg of the glutamic acid, (0.01-8) μg/100 mg of the glycine, and (0.01-0.2) μg/100 mg of the taurine.

18. The natural water-soluble plant compound according to claim 11, comprising (1-200) μg/100 mg of the y-aminobutyric acid, (0.1-50) μg/100 mg of the phenylacetic acid, (0.1-20 μg/100 mg of the glutamic acid, (0.01-8) μg/100 mg of the glycine, and (0.01-0.2) μg/100 mg of the taurine.

19. The natural water-soluble plant compound according to claim 12, comprising (1-200) μg/100 mg of the y-aminobutyric acid, (0.1-50) μg/100 mg of the phenylacetic acid, (0.1-20) μg/100 mg of the glutamic acid, (0.01-8) μg/100 mg of the glycine, and (0.01-0.2) μg/100 mg of the taurine.

20. A drug or a health care product for treating insomnia and/or depression, comprising the natural water-soluble plant compound according to claim 8.
